(19) United States
(12) Patent Application Publication
(10) Pub. No.: US 2005/0010172 A1
(43) Pub. Date: Jan. 13, 2005

(54) Diffusion balloon aortic cannula

(76) Inventor: Robert D. Foster, Rancho Palos Verdes, CA (US)

Correspondence Address: FULBRIGHT AND JAWORSKI L L P;PATENT DOCKETING 29TH FLOOR, 865 SOUTH FIGUEROA STREET, LOS ANGELES, CA, 900172576, US

(21) Appl. No.: 10/734,729

(22) Filed: Dec. 12, 2003

Related U.S. Application Data
(60) Provisional application No. 60/483,329, filed on Jun. 27, 2003.

(51) Int. Cl. A61M 29/00

(52) U.S. Cl. 604/96.01

(57) ABSTRACT

An aortic cannula (10) includes an enclosure (12) which may be affixed to the end of the cannula (10) by means of adhesive or may be embedded in the cannula during layered construction thereof. The enclosure (12) expands with flow through the lumen of the cannula (10) such that the flow area through the material of the enclosure is several times the flow cross-sectional area of the cannula lumen.

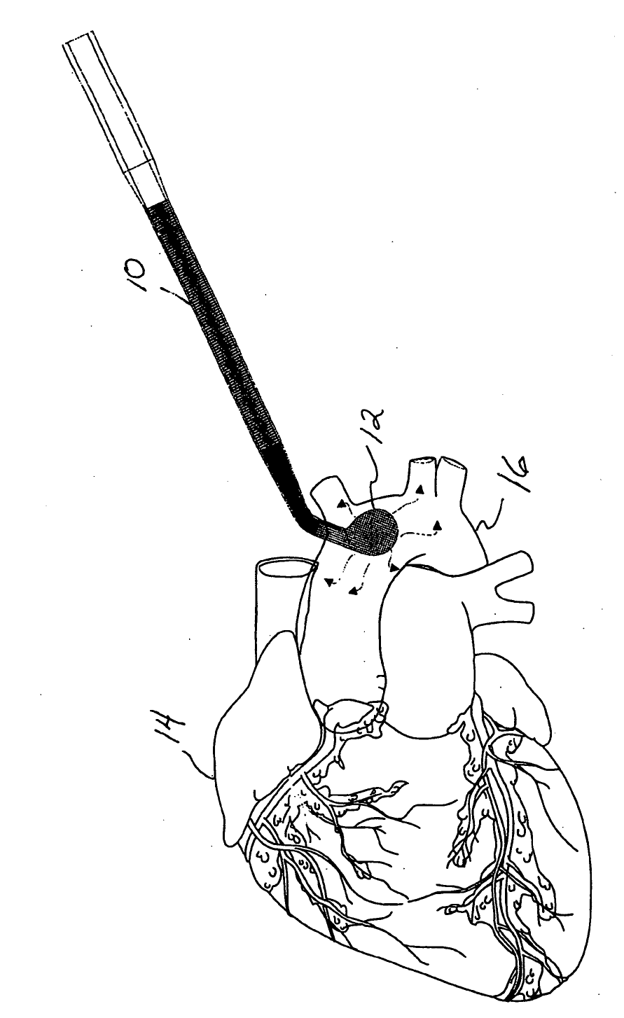

### REFERENCE TO RELATED APPLICATION

This application claims priority to U.S. Provisional Application No. 60/483,329, filed Jun. 27, 2003 to Robert D. Foster, the disclosure of which is incorporated herein by reference.

### BACKGROUND OF THE INVENTION

The field of the present invention is aortic cannula.

The following describes an aortic cannula having an outlet which reduces the flow velocity of fluid, particularly blood, as it exits the lumen of the cannula to avoid dislodging material from the aorta wall. Mechanisms for reducing flow velocity at the tip of an aortic cannula are disclosed in U.S. Pat. Nos. 5,354,288; 5,643,226; 5,685,865; 5,976,114; and 6,059,760. The disclosures of these five patents are incorporated herein by reference.

### SUMMARY OF THE INVENTION

The present invention is directed to an aortic cannula having a porous flexible material forming an enclosure about the terminus of the aortic cannula. The material provides a substantially greater area through which flow passes than the cross-sectional area of the lumen at the terminus of the cannula.

Accordingly, it is an object of the present invention to provide an improved aortic cannula. Other and further objects and advantages will become apparent from the following.

### BRIEF DESCRIPTION OF THE FIGURE

The FIGURE is a side view of an aortic cannula with an enclosure of porous flexible material over the terminus thereof.

### DETAILED DESCRIPTION OF THE PREFERRED EMBODIMENT

In a preferred embodiment, as illustrated in the FIGURE, an aortic cannula 10 includes an enclosure 12 which may be affixed to the end of the cannula 10 by means of adhesive or may be embedded in the cannula during layered construction thereof. The enclosure 12 expands with flow through the lumen of the cannula 10 such that the flow area through the material of the enclosure is several times the flow cross-sectional area of the cannula lumen.

The porous flexible material of the enclosure 12 is preferably of knitted nitinol wire but may be of polymer fabric. The wire is typically 16 french to 26 french fabricated into a loose knit. Thus, the wire may be about 0.0005 to 0.0050 inches in diameter. The interstices of the enclosure 12 through the knitting are small enough that resistance is experienced to flow that flow through the porous flexible material is substantially dispersed through the full area of the porous flexible material. Such resistance distributes the flow jetting from the lumen of the cannula 10 across the full surface of the porous flexible material. Naturally, some variation in flow profile will continue to exist with the maximum flow through the material being at the impinged area.

The deployment of the cannula may be-undertaken with the enclosure 12 drawn into the lumen within the cannula 10 in an everted state. A spring wire may be coupled with or simply behind the enclosure 12 in the lumen before deployment such that the wire can be advanced to push the enclosure 12 from the end of the cannula 10. The spring wire may be curved at the end in accordance with conventional practice such that it comes back upon itself in a loop as it leaves the cannula 10.

Location of the aortic cannula 10 for operation is illustrated in association with a heart 14 in the FIGURE. The enclosure 12 is inserted through an incision into the aorta 16 with flow then dispersed through the porous flexible material rather than impinging in a jet directly at the wall of the aorta 16.

Thus, an improved aortic cannula has been described. While embodiments and applications of this invention have been shown and described, it would be apparent to those skilled in the art that many more modifications are possible without departing from the inventive concepts herein. The invention, therefore is not to be restricted except in the spirit of the appended claims.

1. An aortic cannula having a porous flexible material forming an enclosure about the terminus of the aortic cannula, the material provides a substantially greater area through which flow passes than the cross-sectional area of the lumen at the terminus of the cannula.

2. The aortic cannula of claim 1, the porous flexible material having interstices of sufficient resistance to flow that flow through the porous flexible material is substantially dispersed through the full area of the porous flexible material.

3. The aortic cannula of claim 1, the porous flexible material being knitted wire.

4. The aortic cannula of claim 3, the wire being about 16 to 26 french.

5. The aortic cannula of claim 4, the knitted wire being nitinol wire.

6. The aortic cannula of claim 3, the wire being nitinol about 0.0005 to 0.0050 inches in diameter.

7. The aortic cannula of claim 1, the enclosure being made from polymer fabric.

8. An aortic cannula having a porous flexible material forming an enclosure about the terminus of the aortic cannula, the porous flexible material being knitted nitinol wire, the porous flexible material providing a substantially greater area through which flow passes than the cross-sectional area of the lumen at the terminus of the cannula and having interstices of sufficient resistance to flow that flow through the porous flexible material is substantially dispersed through the full area of the porous flexible material.

9. The aortic cannula of claim 8, the wire being about 0.0005 to 0.0050 inches in diameter.
